(19) United States
(12) Patent Application Publication
(10) Pub. No.: US 2003/0139819 A1
(43) Pub. Date: Jul. 24, 2003

(54) Method and apparatus for closing septal defects

(76) Inventors: Nicholas De Beer, Burlingame, CA (US); Martin Dieck, Cupertino, CA (US)

Correspondence Address: Brian M. Berliner, O'MELVENY & MYERS LLP, 400 South Hope Street, Los Angeles, CA, 90071-2899, US

(21) Appl. No.: 10/052,758

(22) Filed: Jan. 18, 2002

(51) Int. Cl. A61F 2/04

(52) U.S. Cl. 623/23.71; 623/904; 606/158

(57) ABSTRACT

Septal defect occluders are disclosed which can be used with a catheter deployment system to occlude a septal defect. The septal defect occluders of the present invention comprise a metallic frame structure that supports a biodegradable member. The frame structure is made from a shape memory metal such as Nitinol. The frame forms two opposing umbrella or disc shaped halves that are connected via a central region. The biodegradable member is attached to the umbrella or disc shaped halves and can be any of numerous biodegradable materials and is preferably a co-polymer of glycolide and lactide. This material initially forms a barrier to blood flow that occludes the defect. Over time, this material is replaced by the body with scar tissue formation and endothelial cells. The metal frame is left coated with the body's own material that blocks the defect.

### FIELD OF THE INVENTION

The present invention relates to an apparatus and method for closing septal defects including patent foramen ovales, atrial septal defects, and ventricular septal defects.

### BACKGROUND OF THE INVENTION

The term “septal defect” generally refers to a defect that passes through a septum (i.e., a thin wall of muscle or other tissue) that divides or separates distinct areas within the body. Such defects can occur either congenitally or by acquisition between chambers of the heart (i.e., atrium or ventricle) or the great vessels causing shunting of blood through the opening.

In the case of the atrium, the presence of a significantly large septal defect can cause blood to shunt across the defect from the right atrium to the left atrium and hence on to the left ventricle, aorta and brain. If the defect is not closed, the risk of stroke is increased. Studies have shown that adults with strokes of unknown origin (i.e., cryptogenic strokes) have about twice the normal rate of patent foramen ovales than adults with closed foramen ovales. A foramen ovale is a curtain-like opening between the left and right atria. The opening is used during fetal circulation to shunt oxygenated blood away from the lungs because the lungs of a fetus do not fill with air and do not need much blood flow. Foramen ovales normally close and seal after birth; however, in up to 20% of the population the foramen ovale remains open (i.e., patent) and can cause problems later in life.

Shunting of blood from the left heart to the right heart can also have negative consequences that can lead to cardiac failure, pulmonary hypertension and even death. In patients with significant sized ventricular septal defects or patent ductus arteriosus, there is shunting of blood from the high pressure left ventricle or aorta, into the right side chambers and pulmonary arteries that normally have much lower pressures. The increase in blood flow at a high pressure can lead to cardiac failure and death, apart from the serious long-term complication of high pulmonary pressures that can cause a reversal of the direction of the shunt.

A traditional treatment for atrial septal defects is open-heart surgery in which the surgeon opens the chest of a patient and bypasses the heart temporarily (e.g., using a mechanical heart or a “heart-lung machine”). The surgeon then physically cuts into the heart and sutures small defects closed. In the case of larger defects, a patch of a biologically compatible material would be sewn onto the septum to cover (or “patch”) the defect. In order to avoid the morbidity, mortality and long recovery times associated with open-heart surgery, a variety of trans-catheter closure techniques have been attempted. In such techniques, an occluding device is delivered through a catheter to the septal defect site. Once the closure device is positioned adjacent the defect, it must be attached to the rest of the septum in a manner that permits it to effectively block the passage of blood through the defect.

Most trans-catheter systems are mechanically complex and require a great deal of remote manipulation for deployment or retrieval. This extensive remote manipulation, such as by applying tension to one or more cables in order to deploy or to anchor the device in place, not only increases the difficulty of the procedure, but tends to increase the likelihood that the device will be improperly deployed. This can necessitate either retrieval or repositioning so as to effectively occlude the defect and minimize the risk of embolization. Additionally, most of these devices have two separate members joined to each other at a single central interface. With such a device, when the left atrial member is opened, the central point tends to ride to the lower margin of the defect. Another complication with previous trans-catheter devices is that they leave a large amount of foreign material in the heart. This material can cause thrombosis, embolisms, and provide a site for bacterial growth that can lead to bacterial endocarditis and sepsis.

It is desirable, therefore, to provide a simple, collapsible compact closure device that can be delivered through a small catheter. It is also highly advantageous to have such a device that can be readily reversibly deployed and retrieved with a minimal amount of remote manipulation and applied force. It is also desirable to provide a biodegradable material on the device that helps clot and tissue formation, and thereby seal the septal defect.

### SUMMARY OF THE INVENTION

A septal defect occluder of the present invention comprises a metallic frame structure that supports a biodegradable/biocompatible member. The frame structure is made from a variety of different materials but is preferably made out of a shape memory metal such as nitinol. The frame structure can be made from a metal tube, a metal sheet, or from a single wire or plurality of wires. If the frame structure is made from a metal tube or a metal sheet, a plurality of slits are created in the tube or sheet to form two opposing halves that are connected via a central region without slits. The two halves form umbrella or disc-shaped structures that support the biodegradable member.

The biodegradable/biocompatible member can be formed of any of numerous biodegradable or biocompatible materials, such a bioabsorbable synthetic polymers and expanded polytetraflouroethlylene (e-PTFE). Presently the preferred material is a co-polymer of glycolide and lactide. This material will initially form a barrier to blood flow occluding the defect. Over time, this material is replaced by the body with scar tissue formation and endothelial cells, leaving the metal frame coated with the body's own material that blocks the defect.

### BRIEF DESCRIPTION OF THE DRAWINGS

The understanding of the present invention will and the various aspects thereof will be facilitated by reference to the accompanying drawing, submitted for illustration only and not to limit the scope of the invention, where similar reference numerals are used for similar elements in the respective drawings, in which:

FIG. 1 is a schematic of a heart with an atrial septal defect and a ventricular septal defect;

FIG. 2 is a schematic of the heart of FIG. 1 in which the atrial and ventricular septal defects are occluded with septal defect occluders of the present invention;

FIG. 3 is a perspective schematic view of a metal hypotube used in the formation of the metal frame of an occluder of the present invention;

FIG. 4 is a perspective schematic view of the metal hypotube of FIG. 3 with a plurality of slits;

FIG. 5 is a front view of the metal hypotube of FIG. 4 that has been heat-shaped into an occluding device with two umbrella-shaped halves;

FIG. 6 is a top view of a metal sheet that is used in the formation of an alternate frame for an occluder of the present invention;

FIG. 7 is a top view of the metal sheet of FIG. 6 with a plurality of cuts;

FIG. 8 is a perspective view of the metal sheet of FIG. 6 that has been rolled into a tube shape;

FIG. 9 is a perspective view of a further metal frame embodiment of an occluder of the present invention that comprises a plurality of wires;

FIG. 10 is a top view of the metal frame of FIG. 9 after the wires have been molded into two umbrella halves;

FIG. 11 is a perspective view of a metal hypotube with four slits in each half to form four support arms;

FIG. 12 is a perspective view of a biodegradable tube member that is slid over the metal frame of an occluder of the present invention;

FIG. 13 is a side schematic view of the metal frame of FIG. 11 with the biodegradable member of FIG. 12 over it with the frame in the heat set configuration;

FIG. 14 is a top view of an alternate biodegradable member of the present invention;

FIG. 15 is a perspective view of the metal frame of FIG. 11 with two biodegradable members of FIG. 14 attached to either end of the metal frame;

FIG. 16 is a perspective view of the embodiment of FIG. 15 in the heat set configuration in which the biodegradable members form the outer layers of the occluder;

FIG. 17 is a perspective view of yet a further embodiment of the present invention in which the metal frame of FIG. 11 is provided with a plurality of biodegradable threads attached to the arms of the frame in its heat set configuration;

FIG. 18 is a perspective view of the embodiment of FIG. 17 in which the metal frame is in the conformed tube configuration for insertion in a placement catheter;

FIG. 19 is a schematic view of an occluder of the present invention being placed in an atrial septal defect;

FIG. 20 is a schematic view of the distal end of a delivery system of the present invention;

FIG. 21 is an end view of the delivery system of FIG. 20;

FIG. 22 is a side view of the delivery system of FIG. 20;

FIG. 23 is another side view of the delivery system of FIG. 20;

FIG. 24 is a perspective view of a multilumen delivery catheter of the present invention;

FIG. 25 is a perspective view of the multilumen catheter of FIG. 24 with a retaining wire in the retaining wire lumen;

FIG. 26 is a schematic view of a heart with a septal defect with the delivery system being advanced to the defect;

FIG. 27 is a schematic view of a heart with a septal defect with the delivery system delivering an occluder of the present invention to the defect;

FIG. 28 is a schematic view of a heart with a septal defect with the delivery system advancing through the defect to deploy the distal half of the occluder in the left side of the heart;

FIG. 29 is a schematic view of a heart with a septal defect with the delivery system advancing through the defect to deploy the proximal half of the occluder in the right side of the heart;

FIG. 30 is a schematic view of a heart with a septal defect with the retaining wire and guide wire of the delivery system retracted into the multilumen catheter;

FIG. 31 is a schematic view of a heart with a septal defect with the multilumen catheter being retracted into the sheath catheter leaving the occluder of the present invention in place to occlude the defect;

FIG. 32 is a front view of a distal end of a biodegradable/biocompatible member of the present invention with a slit for the guide wire;

FIG. 33 is a rear view of a proximal end of a biodegradable/biocompatible member of the present invention with a hole for the multilumen catheter;

FIG. 34 is a side view of the biodegradable/biocompatible member of FIGS. 32 and 33;

FIG. 35 is a perspective view of yet a further support member embodiment of the present invention;

FIG. 36 is a front view of another embodiment of a support member of the present invention;

FIG. 37 is a front view of yet another embodiment of a support member of the present invention;

FIG. 38 is a perspective view of still another embodiment of a support member of the present invention;

FIG. 39 is a perspective view of a portion of a delivery system for the support member of FIG. 38;

FIG. 40 is a perspective view of the delivery system of FIG. 39 being deployed by a catheter; and

FIG. 41 is a perspective view of another embodiment of a support member of the present invention.

### DETAILED DESCRIPTION OF THE PREFERRED EMBODIMENTS

The present invention satisfies the need for a septal defect occluder for use in closing septal defects via a percutaneous catheter approach. The present occluder device provides a simple, collapsible compact closure device that can be delivered through a small catheter. The occluder device can be readily reversibly deployed and retrieved with a minimal amount of remote manipulation and applied force, and has a biodegradable material that promotes tissue formation to thereby seal the septal defect.

FIG. 1 shows a heart having multiple septal defects. As would be appreciated by a person skilled in the art, a patient's heart may have only one defect or may have multiple defects depending on the cause. Between the right atrium (RA) and the left atrium (LA) is an atrial septal defect (ASD). This defect could be a patent foramen ovale, a congenital septal defect, or any other kind of septal defect. Similarly, between the right ventricle (RV) and the left ventricle (LV) is a ventricular septal defect (VSD).

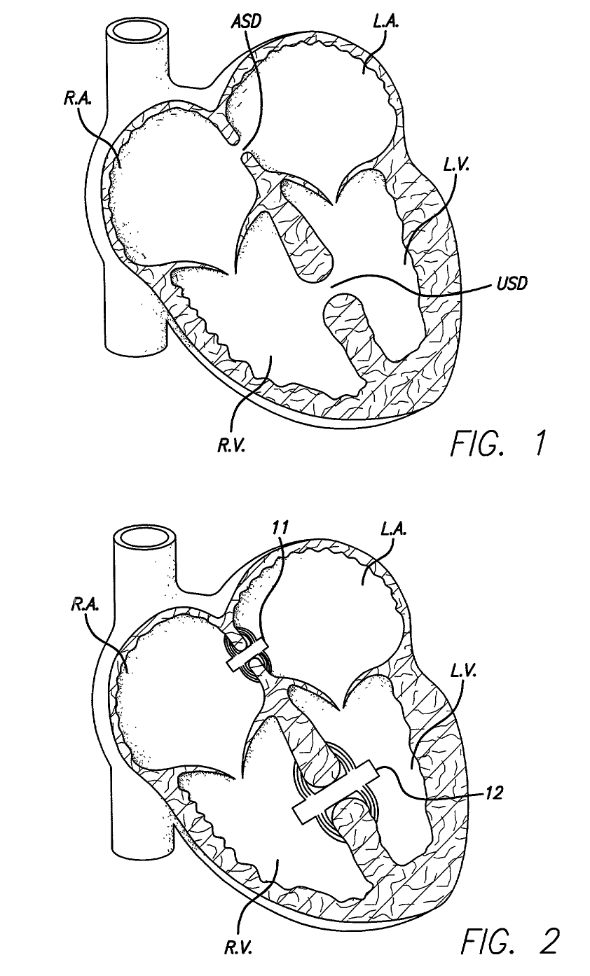

Turning now to FIG. 2, the defects have been occluded with septal defect occluders 11, 12 in accordance with the present invention. The septal defect occluders 11, 12 each comprise a frame structure that supports a biodegradable/biocompatible member. Numerous different embodiments of frame structures are contemplated for use in the septal defect occluders that can support a variety of biodegradable/biocompatible members, as will be described in further detail below.

The frame member can be formed from a variety of materials including metals, plastics including many different plastic polymers, or polymer coated metals. It is preferred that the materials have shape memory such that when they are deformed from a set shape, the material will naturally reform its original shape when no longer deformed. Without limitation, the currently preferred shape memory material of the present invention is a nickel titanium stoichiometric metal alloy commonly referred to as Nitinol or NiTi. Nitinol alloys are preferred because they display shape memory and are also super-elastic in that they can be deformed numerous times and to a great extent without limiting their ability to return to the heat set configuration. Nitinol has also been widely used in numerous medical devices and has been widely studied. Notwithstanding the advantages of Nitinol, other suitable metals exists such as nickel-based high temperature superalloys such as Hastelloy; nickel-based heat treatable alloys such as Incoloy; cobalt-based thermal expansion alloys such as Elgeloy; numerous stainless steel alloys; and other suitable metals known in the art. Additionally, polymers such as polyimide and polymer coated metals can also be used.

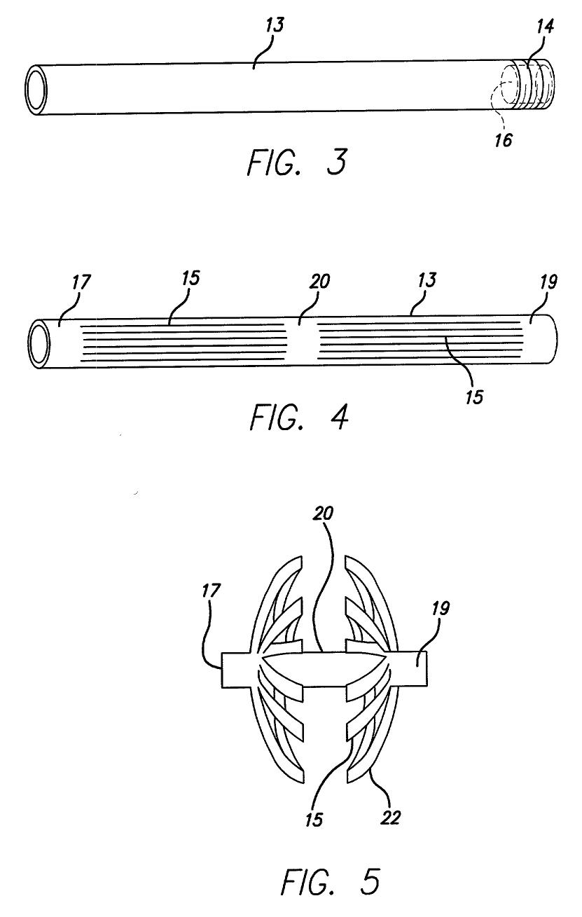

Turning now to FIGS. 3-5, an exemplary frame structure of the present invention is illustrated. The frame structure is formed from a thin-walled metal tube 13 commonly referred to as a hypotube. The thickness of the metal tube can vary from about 0.0025 inches to about 0.010 inches. The inner diameter of the tube can vary from about 0.017 inches for a 0.014-inch guide wire system to about 0.040 inches for a 0.035-inch guide wire system. The length ranges from about 0.5 inches to about 3.0 inches. At one end of the metal tube, an attachment structure is provided that allows the frame structure to connect to a deployment member. The attachment structure illustrated in FIG. 3 includes a threaded opening 14 that has an optional solid plug 16. The optional plug 16 is used to prevent the creation of a metallic shunt after the septal defect is occluded with the septal defect occluder. Alternatively, as described below, a biodegradable/biocompatible member that covers the arms of the septal defect occluder can be used to plug the central hole.

A plurality of longitudinal slits 15 is provided along the length of the metal tube 13 (see FIG. 4). The slits are formed at two distinct areas of the tube 13 leaving a center area 20 of the tube without slits. The slits 15 extend in a direction along the axis of the tube (as illustrated), or they can follow a slight arcuate shape that facilitates the deployment of tile occluder during the closing procedure (discussed below). A distal end 17 and a proximal end 19 of the tube are also left without slits. This way, the ends and the center of the occluder remain tube-shaped after the occluder is heated and its shape is set, leaving three areas with a small diameter and two areas with a larger diameter for occluding the defect (see FIG. 5). The width of the slits can vary from about 0.0015 inches to about 0.025 inches. The number of slits can also vary from about four slits to about thirty slits creating about four to about thirty arms on each half of the occluder. The slits could begin about 0.1 inches to 0.25 inches from the proximal and distal ends of the tube 13. The center region would range from about 0.1 inches to 0.5 inches in width.

The slits are preferable formed using a cutting laser, such as an yttrium aluminum garnet (YAG) laser. It should be appreciated that other cutting lasers well known in the art could also be used. The slits could also be formed using conventional machining techniques, such as grinding or mechanical cutting, electron discharge machining, and the like.

In order to facilitate the placement of the occluder in the heart in a septal defect, the occluder or parts of it can be made radiopaque. There are many known methods of making metal radiopaque, one such method is by coating the metal with vapor-deposited gold or platinum. Another common method is to provide radiopaque bands along the device. For example, two bands can be placed at the distal end and one band at the center and another band at the proximal end. Other methods of making the occluders or parts thereof radiopaque could also be used.

Once the slits are made in the metal tube, the tube is shaped into an occluder of the present invention by providing two opposing umbrellas or discs. In the illustrated embodiment of FIG. 5, the occluder is shaped into two umbrella shaped halves, i.e., a proximal half and a distal half. The umbrellas are created by enlarging the slits 15 to form arms 22 that extend radially along the occluder. It may be desirable to form two flat disc-shaped halves instead of two umbrella-shaped halves. If this is the case, then the arms would fold back on themselves to form arms that point radially outwardly roughly 90° from the tubes axis. The occluder is then placed in a fixture (either umbrella-shaped or disc-shaped) and heat treated to set the shape memory of the metal material. Depending on the type of metal used and the thickness of the metal, different temperature and time duration can be used to set the metal into shape. Setting Nitinol metal into shape is well known in the art. Typically for Nitinol, temperatures between about 500° F. to about 1,100° F. are used and time duration from about one minute to about one hour are used.

The embodiment illustrated shows symmetrical halves, with two umbrella or disc shaped members separated by a center piece. It may be advantageous to have the two umbrella halves be of differing sizes. To accomplish this, longer slits are made in one half of the tube relative to the other half. By varying the slit lengths, the size of the resulting umbrella or disc can vary. For atrial septal defects, it is anticipated that the size of the resulting umbrellas range from about 0.25 inches for small defects up to about 2.0 inches for large defects. For ventricular septal defects, it is anticipated that the size of the resulting umbrellas also range from about 0.25 inches for small defects up to about 2.0 inches for large defects. It is contemplated as part of the invention that occluders be provided in a plurality of different sizes, thereby enabling the medically trained user to select an appropriate size to occlude the defect for the particular patient based on clinical data and imaging data.

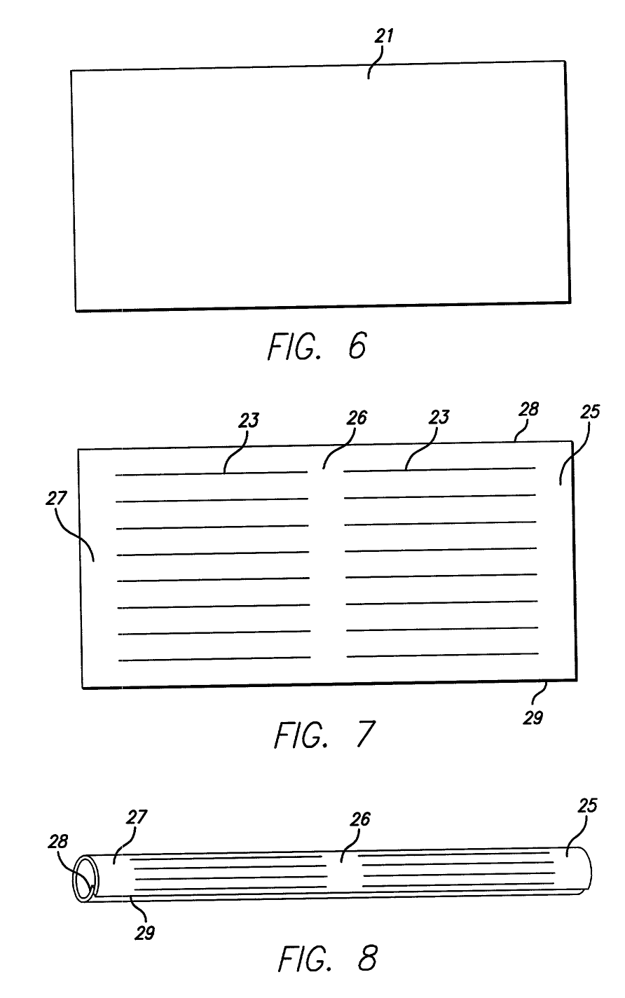

Turning to FIGS. 6-8, another method of making a suitable frame structure is illustrated. The frame is made from a sheet 21 of metal having the same thickness as the metal tube and would range in size from about 0.2 inches to about 2.0 inches in either dimension. The metal would be a shape memory metal as described above. In the preferred embodiment, the metal is Nitinol. A plurality of slits 23 are cut into the metal sheet. As in the device described above, enough slits are made to create four to thirty arms on each side. The slits can be provided parallel to the length of the sheet or can be formed with a slight arcuate shape. The slits can be cut using a laser, electron discharge machining, cutting, etching, or other suitable technique well known in the art. Alternatively, the sheet can be etched using etching techniques well known in the art. One such etching company is VACCO Industries, Inc. located in El Monte, Calif. As with the metal tube, the slits are made leaving a distal end 27, a center region 26, and a proximal end 25. After the slits are formed, the sheet 21 is rolled into a tube (see FIG. 8) that is similar to the tube embodiment described above. Thereafter, the arms are pulled out and the occluder is placed in a fixture to create two umbrella or disc structures. The rolled metal, tube is heat treated at the appropriate temperature and time duration for the type and thickness of the metal. The occluder or parts thereof can also be made radiopaque by gold or platinum deposition or by the use of radiopaque bands as described above.

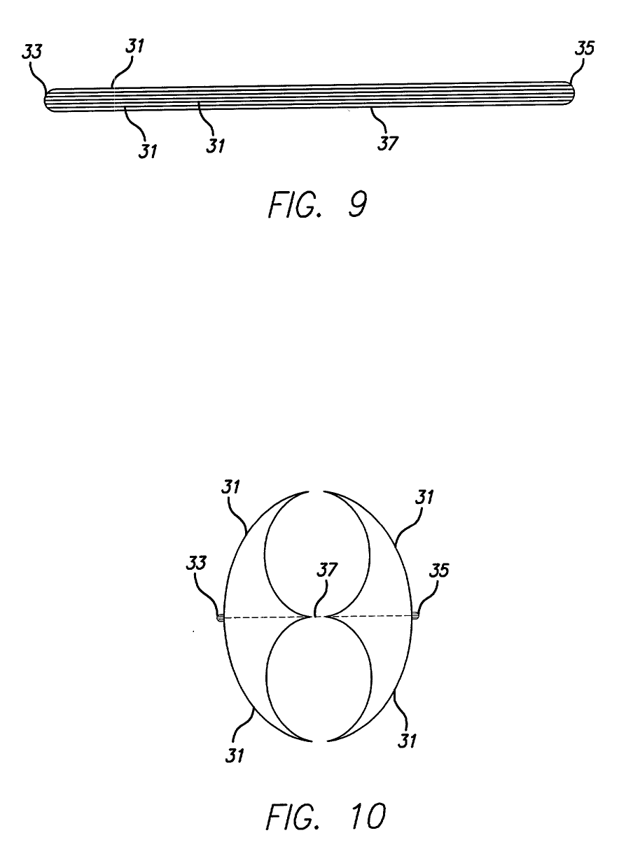

FIGS. 9 and 10 illustrate yet a further frame embodiment of the present invention. In this embodiment a plurality of straight metal wires 31 are used to create the arms of the umbrella or disc shaped structure. The metal wires are held together via two tubular metal clamps, i.e., a distal metal clamp 33 and a proximal metal clamp 35. The clamps are preferably made out of a radiopaque material such as platinum and the like. The metal wires can be made out of any of the suitable shape memory metals discussed above. In the preferred embodiment, Nitinol is used to form the wires and platinum end clamps are used to provide radiopaque markers. Alternatively, platinum coils could be wrapped around the metal frame to provide the needed radiopacity. The wires can vary in thickness from about 0.0007 inches to about 0.015 inches depending on the stiffness of the occluder desired. The wires are then formed within a shaping fixture to form two umbrella or disc shaped halves and the wires form arms for supporting the biodegradable member as discussed below. The formation would provide a center region 37 with a narrow diameter compared to the proximal and distal enlarged regions. The metal would then be heat treated at the appropriate temperature and time depending on the type of metal and the thickness of the wires.

As mentioned above, the occluders of the present invention have a frame component to them and a biodegradable/biocompatible component to them. The biodegradable component can be a variety of different bioabsorbable materials that are well known in the art. For example, the biodegradable material can be a polymer of glycolide (commonly referred to as polyglycolic acid), a polymer of lactide (commonly referred to as polylactic acid), polycaprolactone, poly(hydroxybuterate), poly(hydroxyvalerate) poly(sebacic acid-hexadecandioic acid anhydride), polyorthoesters, co-polymers of the above (for example poly(galactide-co-lactide) which is commonly referred to a poly glycolic and lactic acid or PGLA), and other biodegradable materials. Many of these biodegradable materials can be purchased from Birmingham Polymers, Inc., Birmingham, Ala. Additionally, biocompatible materials such as polytetraflouroethylene (PTFE) and e-PTFE can be used to make a fabric material that is coated with either a drug or a bioabsorbable layer.

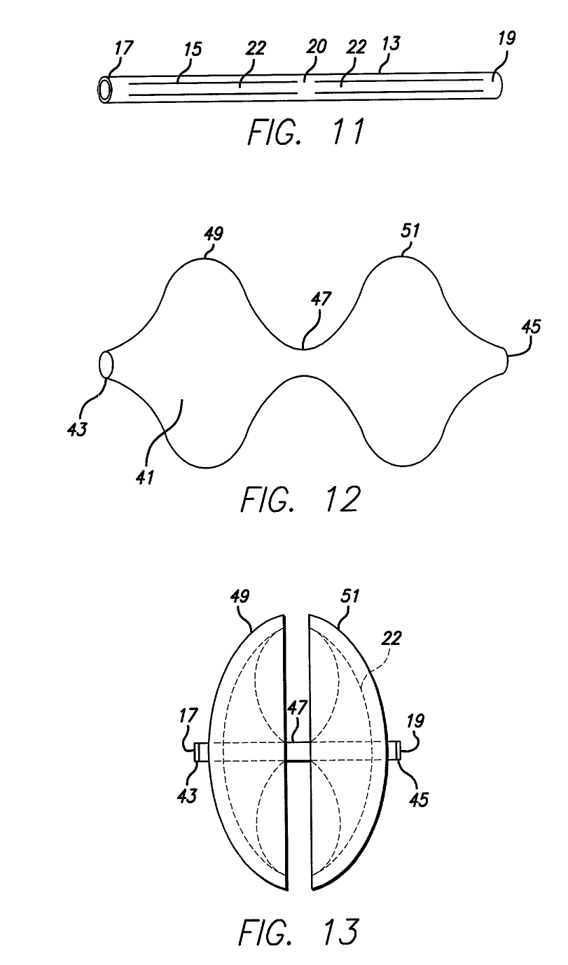

Turning now to FIGS. 11-13, one embodiment of biodegradable/biocompatible member 41 is illustrated with use on a Nitinol tube frame 13. The tube illustrated has four slits 15 in each half to form four arms 22 to support the biodegradable member. The tube has a distal end 17, a center region 20, and a proximal end 19. The biodegradable/biocompatible member has a corresponding distal end 43, a center region 47, and a proximal end 45. Each of these regions is sized to slide over the tube when it is in the tube configuration. The biodegradable/biocompatible member also has an enlarged distal half 49 and an enlarged proximal half 51 which is sized to fit over the arms of the frame when they are in the extended umbrella or disc configuration. Even though a metal tube frame is used to illustrate this biodegradable/biocompatible member and the other biodegradable/biocompatible members discussed below, the same biodegradable/biocompatible members can be used for all the different metal frames discussed.

The enlarged halves of the biodegradable/biocompatible member then form a biodegradable/biocompatible membrane that covers the umbrellas or discs when the occluder is allowed to form its preset shape (see FIG. 13). This biodegradable/biocompatible membrane then provides the structure for the body to slowly degrade and replace with natural scar tissue and endothelial cells once the occluder is in the proper location. The biodegradable/biocompatible member can be made from a sheet of PGLA material, from a braid of e-PTFE, a coil over e-PTFE, or impregnated e-PTFE. The biodegradable/biocompatible member can be attached to the frame by numerous means. The material can be heat bonded to the frame by using an appropriate temperature and time duration for the biodegradable/biocompatible material selected. The temperatures used would be significantly below the heat setting temperatures of the shape memory metal that is used. Alternatively, the biodegradable/biocompatible material can be sutured onto the frame with biodegradable sutures. In the preferred embodiment, the biodegradable/biocompatible tube is adhered to the frame using an adhesive. Currently the preferred adhesive is a biocompatible cyanoacrylate, but other biocompatible adhesives can also be used.

Using the biodegradable/biocompatible material presents a distinct advantage over prior art occluders in that the only part of the device that remains in place long term is the metallic frame and/or a small layer of PTFE or e-PTFE. The rest of the occluder is absorbed by the body and replaced with native cells and tissue. This ensures a permanent closure to the defect and minimizes adverse complications such as bacterial endocarditis and the like.

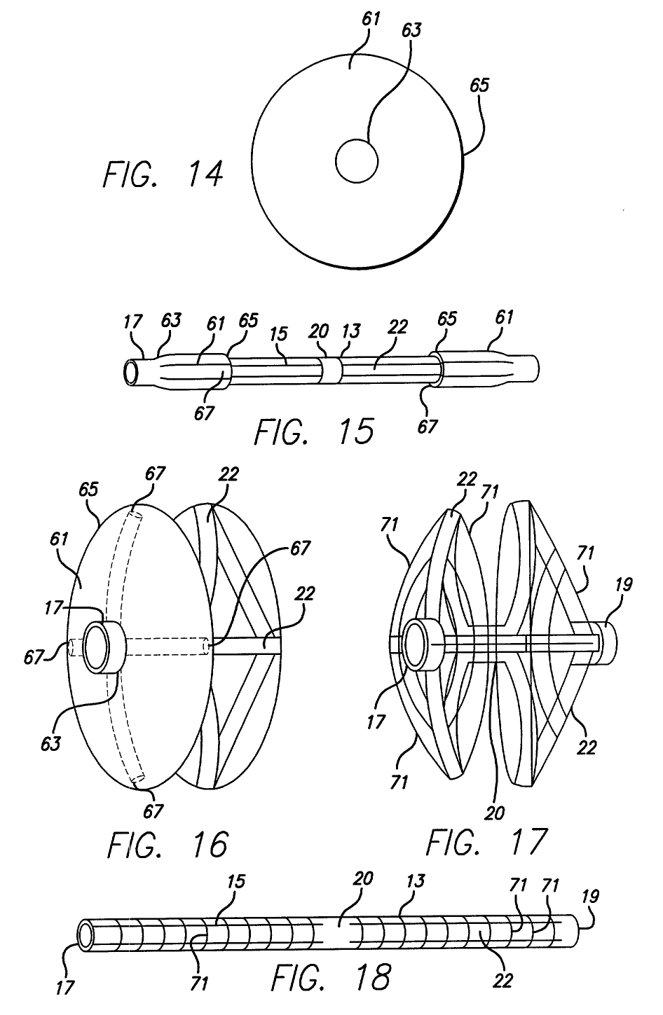

Turning now to FIGS. 14-16, another biodegradable member of the present invention is illustrated with the metal tube frame. Again, this type of biodegradable member can also be used with the other frame embodiments of the present invention. In this embodiment, the biodegradable member comprises two circular sheets of material 61 with an inner hole 63 and an outer circumference 65. One sheet is placed over the distal end 17 of the tube 13, and the other sheet is placed over the proximal end 19 of the tube. The sheet is then fastened to the arms 22 of the umbrellas or discs that form when the device is not constrained via biodegradable sutures 67, heat bonding, or an appropriate adhesive. The outer diameter of the sheet is sized to match the outer diameter of the umbrellas or discs that are formed. The holes 63 of the sheets are sized to fit over the proximal and distal ends of the tube. The diameters of the two sheets can be the same or can vary depending on the size of the two umbrellas or discs.

As is illustrated in the perspective view of FIG. 16, the sheets cover what will become the outer surfaces of the umbrellas or discs once the occluder is in place in a heart of a patient. The inner surfaces will remain with the metal frame and the center 20 of the tube that goes through the center of the defect. When the occluder is constrained, the sheets of the biodegradable material fold up like a closed umbrella for insertion through a catheter.

Turning now to FIGS. 17 and 18, a further biodegradable embodiment is illustrated. In this embodiment, the biodegradable material includes a plurality of biodegradable sutures 71 that are woven onto the metal tube frame illustrated in FIG. 11. As with the other biodegradable members, this member can be used with any of the frames of the present invention. The sutures make a spider web-like around the frame and also can traverse across the frame to fill the center of the frame. The sutures can be circular in diameter and range from about 0.005 inches to about 0.015 inches. Alternatively, the sutures can be ribbon-shaped and have a thickness of about 0.003 inches and a width of about 0.010 inches. The sutures collapse inside the tube when the frame is constrained to fit within a placement catheter (see FIG. 18).

The placement of the device is illustrated in FIG. 19 wherein a catheter 81 is used to deliver the occluder of the present invention. The occluder illustrated is similar to the occluders of FIGS. 11-13, but it should be understood that all embodiments of the occluder of the present invention could be similarly placed. The patient is prepped and access to the right side of the heart is found. Usually access will be through the femoral vein, however, other access points such as the jugular vein or the cephalic vein can also be used. The catheter is then advanced to the right side of the heart.

For closing an atrial septal defect, the catheter is advanced into the right atrium and then though the defect into the left atrium. An appropriate sized occluder of the present invention is then advanced into the proximal end of the catheter using a deployment member (not illustrated). The deployment member (not illustrated) releasably attaches to the proximal end of the occluder. The occluder is then advanced until the distal half of the occluder is deployed in the left atrium. Because the frame of the occluder is a shape memory metal, the half of the occluder that is in the left atrium adopts its preset umbrella or disc shape and the biodegradable material 41 covers the outer surface of the occluder. The catheter and the occluder are then slowly retracted until the umbrella or disc catches onto the outer edges of the defect (as illustrated in FIG. 19). The occluder is then forced further out of the catheter by retracting the catheter while advancing the deployment member. Eventually, the entire occluder is out of the catheter and the proximal half of the occluder can adopt its preset shape. Once proper placement of the occluder is confirmed, the occluder is released from the deployment member and the catheter and the deployment member are removed from the patient leaving the occluder in place around the defect (as shown in FIG. 2).

A similar procedure is used to close a ventricular septal defect except that the catheter is initially advanced through the atrio-ventricular valve of the right ventricle and then passed through the ventricular septal defect into the left ventricle. The occluder is then advanced through the catheter and half of it is deployed in the left ventricle and then slowly retracted until it catches on the edges of the defect. The rest of the occluder is then deployed and forms its shape in the right ventricle and then detached to occlude the defect (as shown in FIG. 2).

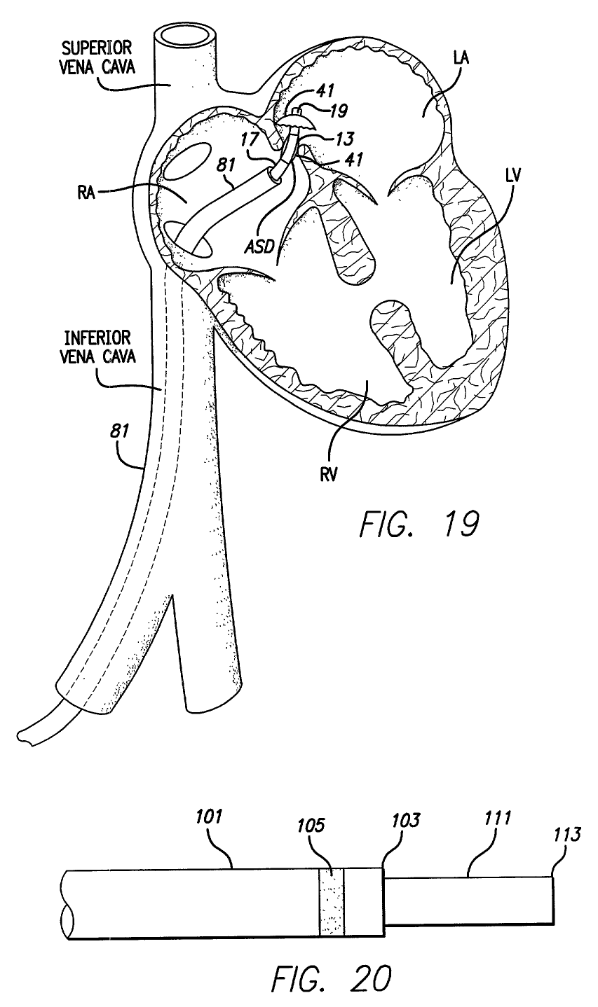

Turning now to FIGS. 20-29 a deployment system of the present invention is illustrated occluding a septal defect. FIGS. 20 and 21 illustrated a delivery system of the present invention. The delivery system comprises an outer sheath catheter 101 ranging in diameter from about 0.060 inches to about 0.10 inches. The length of the sheath catheter ranges from about 80 cm to about 150 cm. The sheath catheter can be any standard sheath catheter in the cardiac catheterization art. Typically these catheters are made of extruded polymers, but other materials well known in the art can also be used. Only the distal end 103 of the sheath catheter is illustrated. Near the distal end of the sheath catheter is a radiopaque marker band 105.

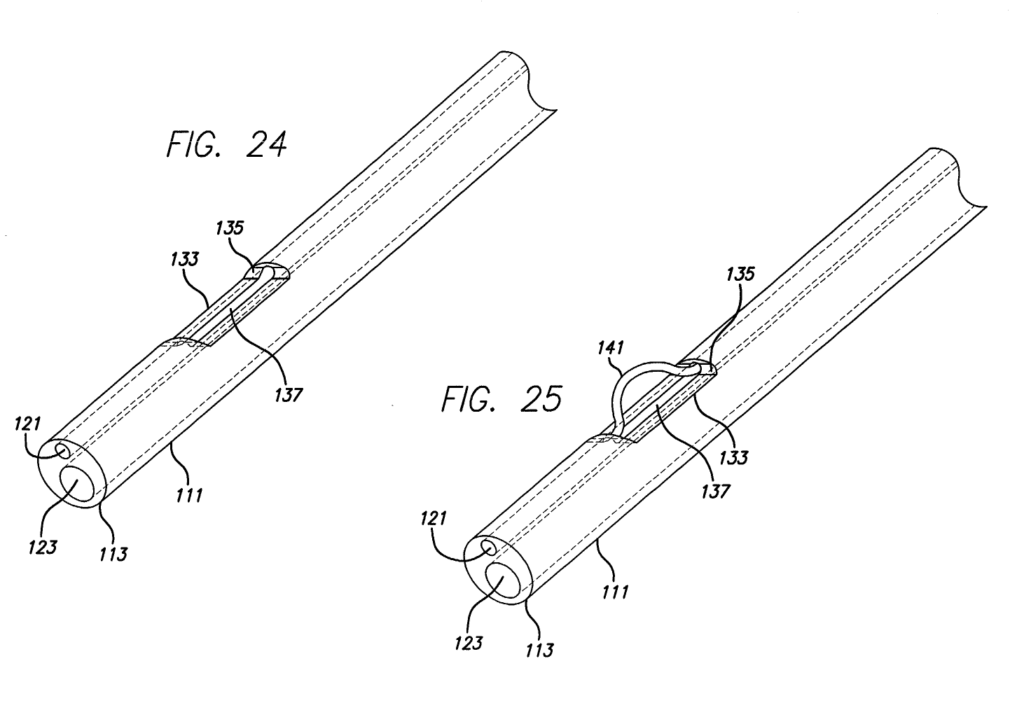

Inside the sheath catheter is a multilumen delivery catheter 111. In the illustrated embodiment, the catheter has two lumens, i.e., a guidewire lumen 123 and a releasable wire lumen 121. Turning now to FIGS. 24 and 25, perspective views of the multilumen catheter of the present invention are illustrated. The diameter of the multilumen catheter can range from about 0.035 inches to about 0.060 inches with 0.042 inches being presently preferred. The length of the delivery catheter can range from about 80 cm to about 150 cm with 120 cm being presently preferred. The delivery catheter can be made of standard catheter materials well known in the art such as extruded polymers. The diameter of the guidewire lumen can range from about 0.016 inches to about 0.020 inches with 0.017 inches being presently preferred. The diameter of the restraining wire lumen can range from about 0.007 inches to about 0.013 inches with 0.010 inches being presently preferred.

A cut-out notch 133 is located about 3 mm to about 10 mm from the distal end 113 of the multilumen catheter, preferably with beveled sides 135, that cuts into the restraining wire lumen 121 leaving a channel 137 in place of the restraining wire lumen. This allows exposure to the restraining wire 141 (see FIG. 25) for access during manufacturing for placing the restraining wire around the outside of the center area 20 of the support member of the present invention. The support member of the present invention is placed over the delivery catheter thorough the center area such that the center area is centered over the cutout notch 133 of the delivery catheter. The restraining wire is then advanced through the proximal end of the restraining wire lumen to the cutout notch. The restraining wire is then placed around the outside of the central area of the support member and then reinserted into the restraining lumen near the distal end of the delivery catheter to hold the support member in place. Finally, a biodegradable/biocompatible member of the present invention is then placed over the distal end of the delivery catheter and then over the support member and then the delivery catheter is advanced into the sheath catheter for delivery into the patient.

The restraining wire can range from about 0.005 inches to about 0.010 inches in diameter with 0.007 inches being presently preferred. The restraining wire can be made out of numerous materials or combinations of materials, such as stainless steel, platinum, Nitinol, and the like. Currently a Nitinol wire is preferred. The length of the wire ranges from about 80 cm to about 150 cm, which are typical lengths of cardiac catheter delivery systems. The wire should be about 5 cm to about 10 cm longer than the delivery catheter for ease in retrieval.

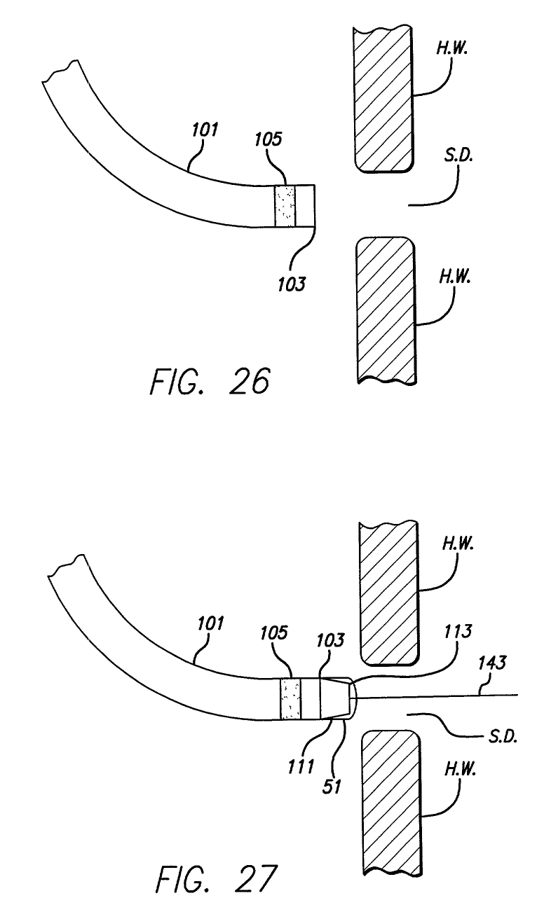

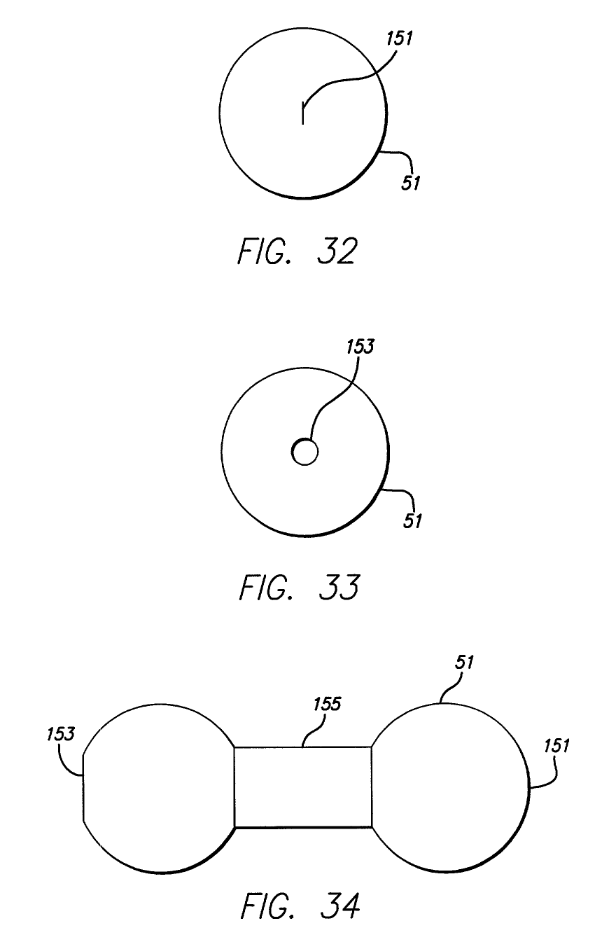

Turning now to FIGS. 26-31, the delivery of an occluder of the present invention to a septal defect (SD) of a patient in a heart wall (HW) is illustrated. The first step is to advance the sheath catheter to the septal defect as described above while locating under fluoroscopy or other imaging device the distal end of the sheath catheter via the marking band (see FIG. 26). The next step is to advance the delivery catheter with the occluder around it into the defect (see FIG. 27). This is usually done via a guidewire 143 that is in the guidewire lumen 123 that extends through a slit 151 (see FIG. 32) in the biodegradable/biocompatible member 51 that covers the occluder.

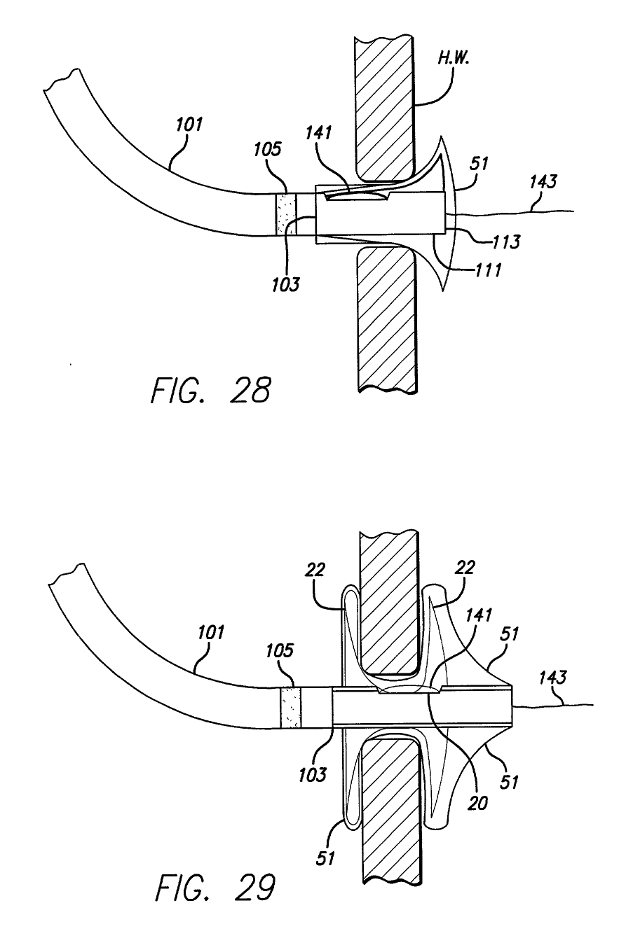

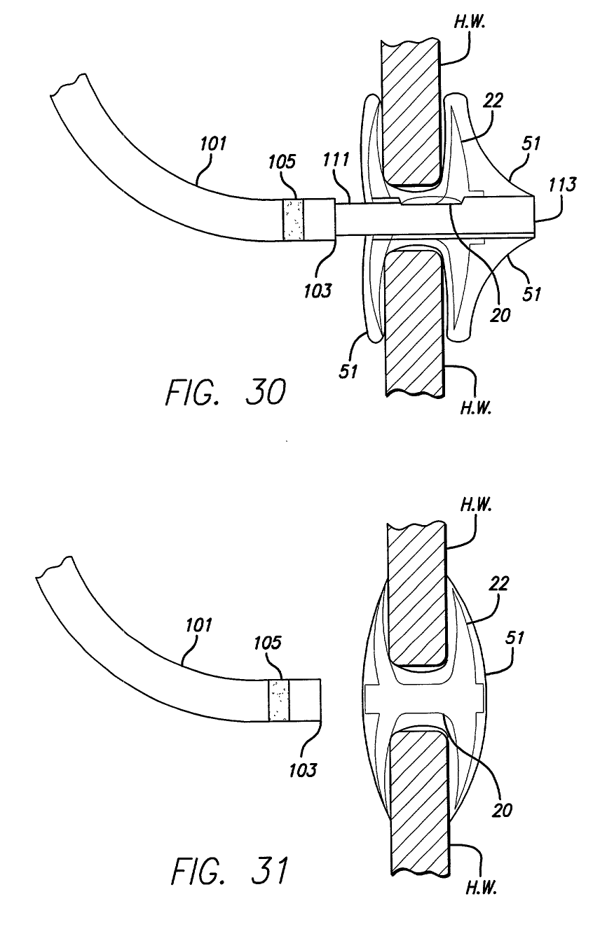

Next, the delivery catheter is slowly advanced until the distal half of the occluder is located in the left side of the heart and the support member forms its natural shape by having the support arms 22 form an umbrella shape (see FIG. 28). The occluder is being kept in place by the restraining wire 141 around the center area 20 of the occluder support member. Next the sheath catheter is retracted to allow the deployment of the distal half of the occluder (see FIG. 29). The location of the occluder is verified using fluoroscopy or other appropriate imaging techniques. Once the location is verified, the guidewire 143 and the restraining wire 141 are retracted into the delivery catheter, with the retaining wire being pulled proximally until the occluder is released in place (see FIG. 30). Next the delivery catheter is retracted into the sheath catheter (see FIG. 31) leaving the occluder in place around the defect. Finally, the sheath catheter and delivery catheter are removed from the patient.

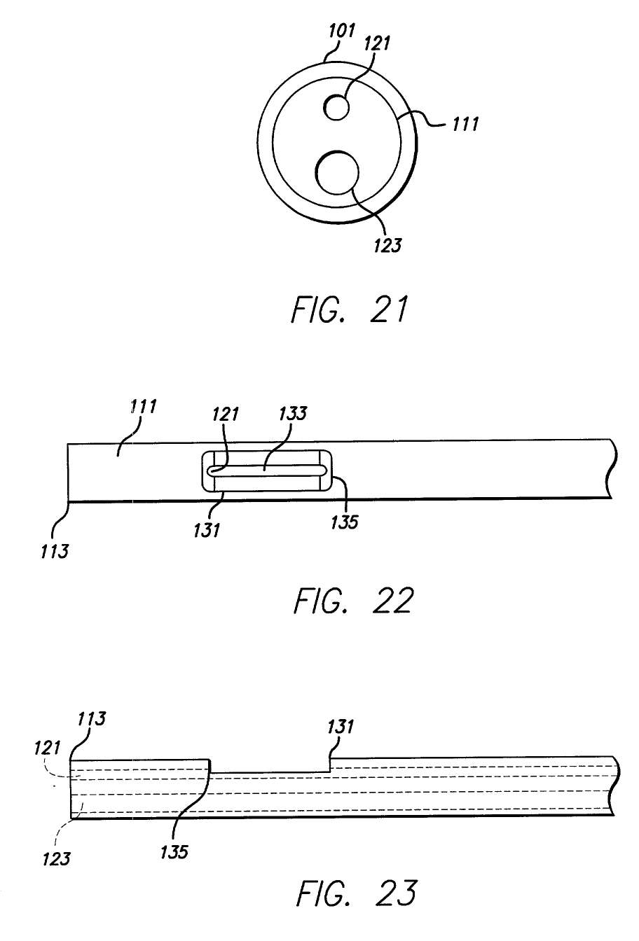

Turning now to FIGS. 32-34 a biodegradable/biocompatible member 51 of the present invention is illustrated for use with the delivery system detailed in FIGS. 22-31. The member has a small slit 151 located at the distal half of the member for the guidewire to be inserted through. Located on the proximal half of the member is a hole 153 for the delivery catheter to be inserted through. As would be apparent to a person skilled in the art, the guidewire slit 151 could be replaced with a small hole and the delivery catheter hole could be replaced with a slit. The member is preferably made out of PGLA, but could be made out of PTFE or e-PTFE as noted above. The member has a cylindrical middle section 155 for centering over the center area of the support member.

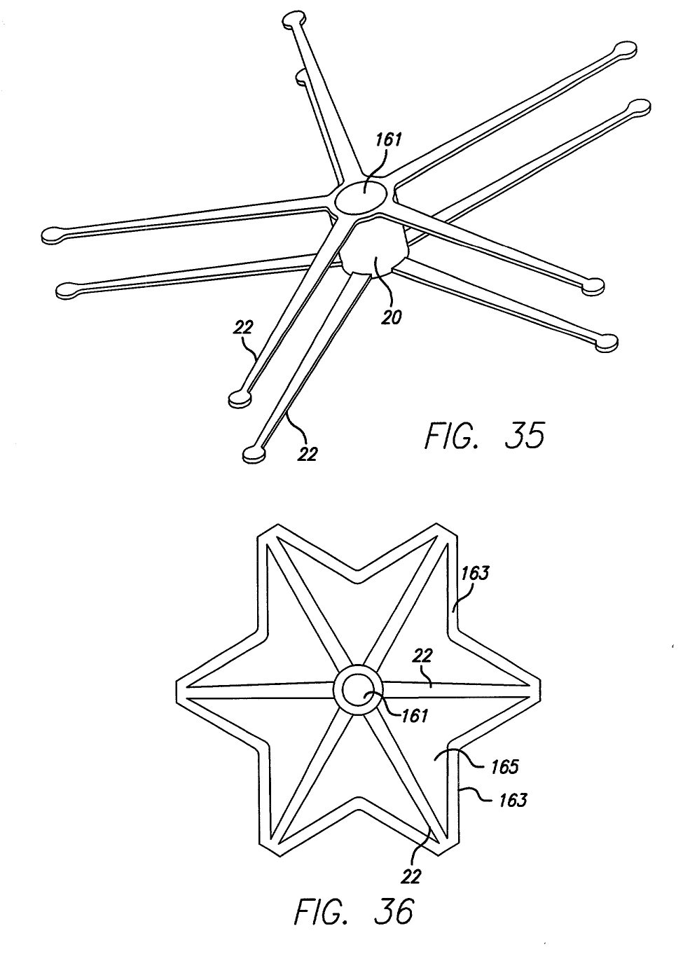

FIG. 35 illustrates yet a further support member embodiment of the present invention. The support member of FIG. 35 is a star shaped pattern with five arms 22 that radiate outwardly from a center tube 20 with an interior lumen 161. As would be apparent to a person skilled in the art, the number of arms in the star pattern can vary. Currently the number varies from about four to about eight arms. The center area 20 is used to secure the support member to the delivery catheter using the restraining wire of the delivery catheter. The center area can vary from about 1 mm to about 5 mm with about 2 mm being presently preferred. The radial length of the arms of the star can vary from about 0.25 inches to about 1.0 inches with about 0.5 inches being presently preferred. The star pattern can be created by cutting a tube and folding the arms back or by etching a sheet and then bonding the star patterns to a cylindrical tube. The preferred material is Nitinol, however, other materials for the support members listed above can be used.

FIG. 36 illustrates another embodiment of a support member of the present invention. In this embodiment the support member forms a star-shaped pattern with six arms 22 that radiate outward from a center region and has six chevron-shaped support arms 163 to help secure the support member. The support member would be similar to the support member of FIG. 33 in that two star-shaped members would be attached to a central cylindrical portion. As would be apparent to a person skilled in the art, the number of arms in the star pattern can vary. Currently the number varies from about four to about eight arms. The center area can vary from about 1 mm to about 5 mm with about 2 mm being presently preferred. The radial length of the arms of the star can vary from about 0.25 inches to about 1.0 inches with about 0.5 inches being presently preferred. The star pattern can be created by cutting a tube and folding the arms back or by etching a sheet and then bonding the star patterns to a cylindrical tube. The preferred material is Nitinol, however, other materials for the support members listed above can be used.

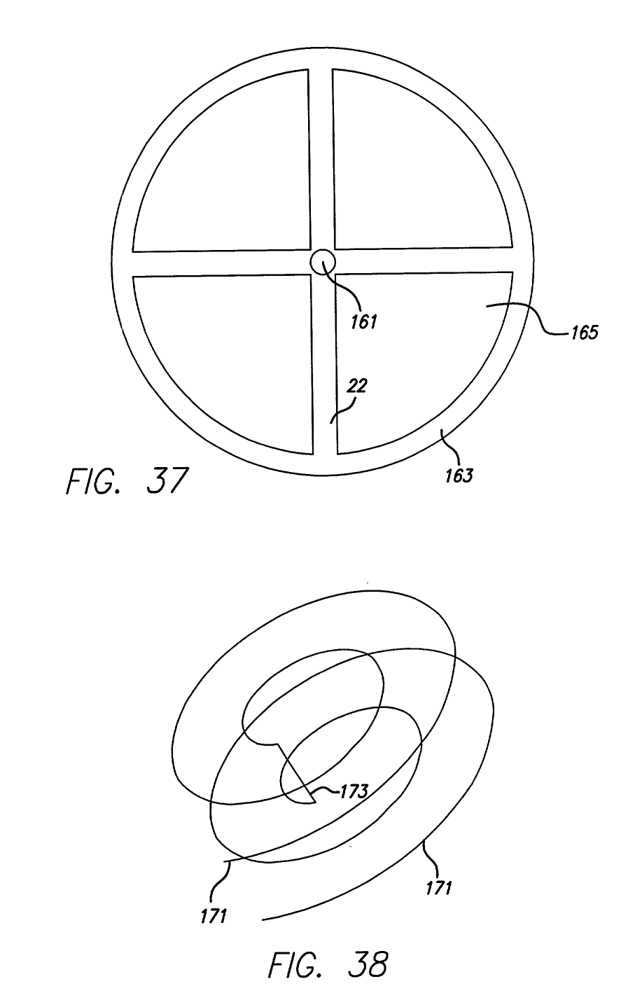

FIG. 37 illustrates yet another embodiment of a support member of the present invention. In this embodiment the support member forms a wheel-like structure with four spokes (arms) 22 radiating outwardly from a center region with a lumen 161. The support member would be similar to the support member of FIG. 35 in that two wheel-shaped members would be attached to a central cylindrical portion. As would be apparent to a person skilled in the art, the number of arms in the wheel pattern can vary. Currently the number varies from about four to about eight arms. The center area can vary from about 1 mm to about 5 mm with about 2 mm being presently preferred. The radial length of the spokes of the wheel can vary from about 0.25 inches to about 1.0 inches with about 0.5 inches being presently preferred. The wheel pattern can be created in the same way as the embodiment of FIGS. 33 and 34 by etching a sheet and then bonding the wheel patterns to a cylindrical tube. The preferred material is Nitinol, however, other materials for the support members listed above can be used.

FIG. 38 illustrates another embodiment of a support member of the present invention. In this embodiment the support structure is made out of a wire that is formed into two spirals 171 with a center connection 173. The wire is preferably Nitinol, however, other materials mentioned above could be used. The center area can vary in length from about 1 mm to about 5 mm with about 2 mm being presently preferred. The spirals can vary in diameter from about 0.5 inches to about 3.0 inches with about 1 inch being presently preferred.

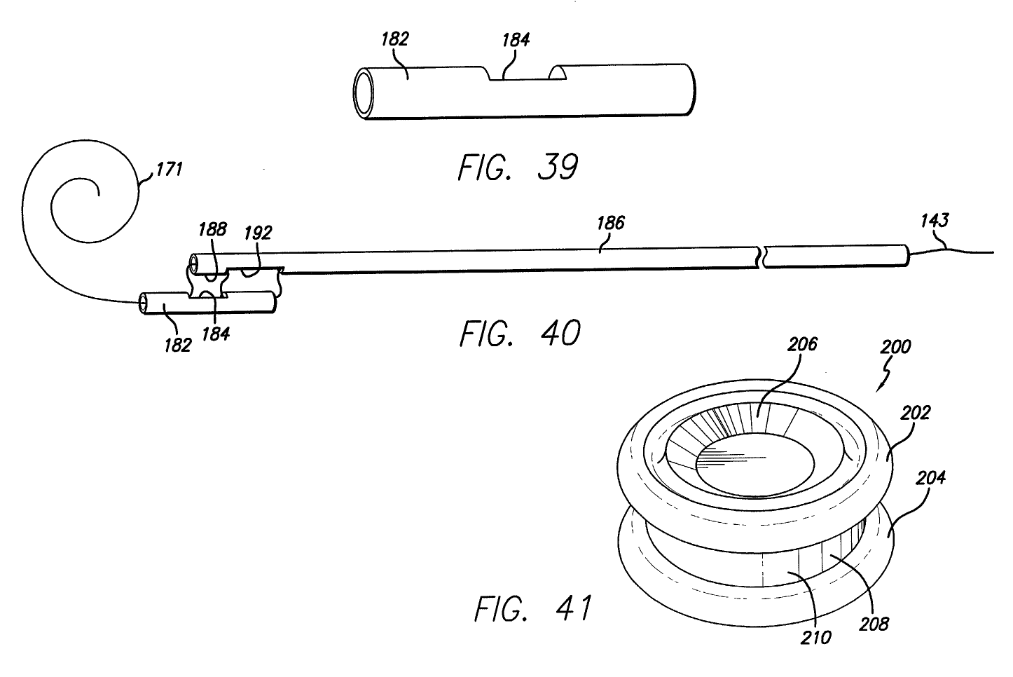

FIGS. 39-40 illustrate a delivery system for use with the support member of FIG. 38. The delivery system includes a first tube 182 having a notch 184. The support member is bonded to an end of the first tube such that the spirals 171 of the support member extend therefrom. FIG. 40 appears to show only one of the spirals 171, but it should be understood that the two spirals would be aligned together from the orientation of FIG. 40. The tube 182 would have an outside diameter that ranges from 0.020 inches to 0.040 inches, a wall thickness ranging from 0.0025 inches to 0.010 inches, and an overall length ranging from 10 mm to 32 mm. A second tube 186 has a notch 192 spaced from a distal end of the tube that defines a key 188. A guidewire 143 extends through the second tube 186 and threads successively through the notch 192 into the first tube 182, then through the notch 184 back into the second tube, and then from the distal end of the second tube back into the notch 192 of the first tube. When the slack is taken out of the guidewire 143, the first and second tubes engage each other with the key 188 inserted into the notch 184. The second tube 186 runs the length of a delivery catheter (not shown) with the guidewire 143 inside the second tube holding the first and second tubes together. By pulling on the guidewire 143 from the proximal end of the delivery catheter, the guidewire 143 releases from the first tube 182 to thereby deploy the support member.

FIG. 41 illustrates yet another embodiment of a support member 200 of the present invention. The support member 200 comprises two rings 202, 204 comprised of ePTFE material filled with an adhesive, such as an adhesive curable with ultraviolet (UV) light. The ring 202 has a membrane 206, and the ring 204 has a membrane 210, which join in the center to provide a common membrane 208 sealing the hole within the rings. Each ring and associated membrane may be assembled from a sheet of ePTFE material having a solid disc shape. The outer edge of the disc is folded over and attached to itself to form the ring. The rings would further include a valve to permit filling of the rings. It is anticipated that the support member be deployed in an unfilled configuration, and the rings would be filled after deployment to lock it permanently in place within a septal defect.

While several particular embodiments of the invention have been illustrated and described, it will be apparent that various modifications can be made without departing from the spirit and scope of the invention. For example, different materials could be used in the different parts of the assemble. Accordingly, it is not intended that the invention be limited except by the following claims.

What is claimed is:

1. A septal defect occluder comprising:
(a) a shape memory frame with a proximal end and a distal end wherein the frame can be constrained to fit within catheter and when not constrained forms two opposing umbrella or disc shaped halves; and
(b) a biodegradable/biocompatible member covering at least a portion of the umbrella or disc shaped halves.

2. The septal defect occluder of claim 1, wherein the frame is made from a metal tube having a plurality of slits.

3. The septal defect occluder of claim 1, wherein the frame is made from a metal sheet having a plurality of slits.

4. The septal defect occluder of claim 1, wherein the frame is made from at least one metal wire.

5. The septal defect occluder of claim 1, wherein the frame is comprised of a Nitinol material.

6. The septal defect occluder of claim 1, wherein the biodegradable/biocompatible member comprises a tube having a small diameter distal end, a small diameter center, and a small diameter proximal end with two larger diameter regions disposed between the center and the proximal and distal ends.

7. The septal defect occluder of claim 1, wherein the biodegradable/biocompatible member comprises a first circular sheet placed over the distal end of the frame and a second circular sheet placed over the proximal end of the frame.

8. The septal defect occluder of claim 1, wherein the biodegradable/biocompatible member comprises a plurality of threads.

9. The septal defect occluder of claim 8, wherein the plurality of threads form a spider web-like structure when the metal frame is not constrained.

10. The septal defect occluder of claim 1, wherein the biodegradable/biocompatible member comprises a copolymer of galactide and lactide.

11. A septal defect occluder comprising:
(a) a shape memory frame having a plurality of arms with a proximal end and a distal end wherein the frame can be constrained to fit within a catheter and when not constrained forms two opposing umbrella or disc-shaped halves, and the metal frame has a releasable attachment means located at the proximal end for attaching to a deployment member;
(b) a biodegradable/biocompatible member covering at least a portion of the umbrella or disc shaped-halves; and
(c) means for fixing the biodegradable/biocompatible member to the frame.

12. The septal defect occluder of claim 11, wherein the frame is comprised of a metal tube having a plurality of slits.

13. The septal defect occluder of claim 11, wherein the frame is comprised of a metal sheet having a plurality of slits.

14. The septal defect occluder of claim 11, wherein the frame is comprised of at least one metal wire.

15. The septal defect occluder of claim 11, wherein the frame is comprised of a Nitinol material.

16. The septal defect occluder of claim 11, wherein the biodegradable/biocompatible member comprises a tube having a small diameter distal end, a small diameter center, and a small diameter proximal end with two larger diameter regions disposed between the center and the proximal and distal ends.

17. The septal defect occluder of claim 11, wherein the biodegradable/biocompatible member comprises a first circular sheet disposed over the distal end of the frame and a second circular sheet disposed over the proximal end of the frame.

18. The septal defect occluder of claim 11, wherein the biodegradable/biocompatible member is comprised of a plurality of threads.

19. The septal defect occluder of claim 18, wherein the plurality of threads form a spider web-like structure when the metal frame is not constrained.

20. The septal defect occluder of claim 11, wherein the biodegradable/biocompatible member is comprised of a copolymer of galactide and lactide.

21. A septal defect occluder comprising:
a support member having a first inflatable ring, a second inflatable ring and a membrane joined to said first and second rings at a common center thereof; and
an adhesive material adapted to fill said first and second rings upon deployment of said support member in a septal defect.

22. The septal defect occluder of claim 21, wherein said support member is comprised of a biodegradable/biocompatible member.

23. A method of occluding a septal defect comprising the steps of:
(a) accessing the right side of the heart via a catheter;
(b) advancing the catheter through a septal defect;
(c) advancing a septal defect occluder having proximal and distal ends with a shape memory frame and a biodegradable/biocompatible member through the catheter;
(d) allowing the distal end of the occluder to form a preset shape in the left side of the heart;
(e) withdrawing the catheter and the occluder slowly until the distal end contacts the heart tissue around the opening of the defect;
(f) withdrawing the catheter until the occluder is fully deployed in the heart and the proximal end has formed its preset shape;
(g) removing the catheter from the patient;
(h) allowing the body to degrade the biodegradable member and cover the frame with native tissue.
